(19) United States
(12) Patent Application Publication
(10) Pub. No.: US 2023/0000852 A1
(43) Pub. Date: Jan. 5, 2023

(54) Treatment of Dermal Cytokine Storm Syndromes

(71) Applicant: Bridge Pharma, Inc., Lakewood Ranch, FL (US)

(72) Inventors: A.K. Gunnar Aberg, Lakewood Ranch, FL (US); Kresimir Pucaj, Toronto (CA)

(21) Appl. No.: 17/851,165

(22) Filed: Jun. 28, 2022

Related U.S. Application Data
(60) Provisional application No. 63/216,293, filed on Jun. 29, 2021.

(51) Int. Cl. A61K 31/4535; A61P 29/00

(52) U.S. Cl. CPC A61K 31/4535; A61P 29/00

(57) ABSTRACT

Described herein are methods of treating human and canine patients suffering from dermal Cytokine Storm syndromes related to dermal diseases such as atopic dermatitis, psoriasis or urticaria. A method of reducing the incidence and/or the intensity of a symptom of dermal hypercytokinemia in a patient suffering from an underlying dermal condition who develops the dermal hypercytokinemia as a consequence of the underlying dermal condition includes administering to the patient a therapeutically effective amount of norketotifen an isomer, an isomeric mixture, a prodrug or a pharmaceutically acceptable salt thereof. The symptom of dermal hypercytokinemia is an eruption of new dermal lesions accompanied by intense lesional and/or non-lesional pruritus.

### TECHNICAL FIELD

The embodiments disclosed herein relate to methods for the treatment of dermal hypercytokinemias with RS-norketotifen (NK).

### BACKGROUND

The term “cytokine storm” refers to the simultaneous release of multiple cytokines by the immune system and is most often triggered by a microbial infection. There is currently an increased interest in the cytokine storm syndrome. Thus, a Google search in 2011 resulted in 170,000 hits for cytokine storm, while a Google search for “cytokine storm” in 2021 gave about 6.9 million hits. A cytokine storm is not a disease in itself, but is a syndrome, which is an endpoint of different initial insults, most often by microbes, such as bacteria, fungi or viruses. The term “hypercytokinemia” is herein used synonymously with the term “cytokine storm syndrome” or just “cytokine storm.” Dermal hypercytokinemias refer to severe forms of dermal diseases that have demanded corticosteroidal rescue medication. Hypercytokinemias are caused by the immune system after a group of germ-line-encoded proteins, named pattern-recognition receptors, initiate an uncontrolled and excessive release of pro-inflammatory cytokines in response to the initial microbial or allergic insult.

A hypercytokinemia can be systemic, but is most often local, as usually is the case for patient suffering from dermal diseases, such as atopic dermatitis, psoriasis and chronic urticaria. Symptoms of systemic hypercytokinemia include fever, widespread pain, shortness of breath, low blood pressure and high heart rate. The laboratory findings usually include abnormally high liver enzymes, elevated inflammatory disease markers like TNFα and/or IL-6 and high ferritin levels. It has been suggested that high ferritin levels may be used as a litmus test for systemic cytokine storm.

No universally recommended treatment exists for patients with hypercytokinemia. Current experimental use includes the administration of a corticosteroid, cyclosporine and the IL-6 inhibitor tocilizumab, which is an injected monoclonal antibody, irreversibly targeting the interleukin-6 receptor (IL-6R) for up to several weeks.

What is needed for the treatment of hypercytokinemia are orally, topically and parenterally active drugs that simultaneously inhibit the release of the cytokines and chemokines that cause the often fatal condition of hypercytokinemia. Chemokines are often referred to as being types of cytokines. For the sake of simplicity, chemokines are therefore in this document often referred to as cytokines. As examples, the chemokines MCP-1 and MIP-1α are referred to herein as cytokines.

### SUMMARY

In an aspect, a method of reducing the incidence and/or intensity of a symptom of dermal hypercytokinemia in a patient suffering from an underlying dermal condition who develops the dermal hypercytokinemia as a consequence of the underlying dermal condition comprises orally administering, intravenously administering, subcutaneously administering, intramuscularly injecting, topically administering or dermally administering to the patient a therapeutically effective amount of norketotifen as a free base, an isomer, an isomeric mixture, a prodrug or a pharmaceutically acceptable salt thereof, wherein the symptom of dermal hypercytokinemia is an eruption of new dermal lesions accompanied by intense lesional and/or non-lesional pruritus.

### DETAILED DESCRIPTION

As reported herein, norketotifen (NK) has now been found to inhibit the release of numerous mediators—cytokines and chemokines—from various granulocytes, such as white blood cells. The term granulocyte, as used herein, refers to a cell with secretory granules in its cytoplasm, i.e. neutrophils, basophils, eosinophils, mast cells and keratinocytes.

Described herein are methods of treating dermal hypercytokinemia, specifically reducing the incidence and/or intensity of dermal hypercytokinemia or a symptom thereof, in a patient suffering from an underlying dermal condition such as a dermal viral, bacterial, mold or fungal infection, atopic dermatitis, psoriasis or chronic urticaria, the method comprising administering, to the patient a therapeutically effective amount of norketotifen, an isomer, an isomeric mixture, a prodrug, or a pharmaceutically acceptable salt thereof. In an aspect, the symptom of dermal hypercytokinemia is an eruption of new dermal lesions accompanied by intense lesional and/or non-lesional pruritus.

As used herein, dermal hypercytokinemia is a local hypercytokinemia caused by a local release of cytokines in or to the skin. Dermal hypercytokinemia is typically characterized by an eruption of new dermal lesions and by intense lesional and/or non-lesional pruritus. Dermal hypercytokinemia causes a significant reduction in quality of life, often in the form of unbearable pruritus.

In an aspect, the patient has not been previously treated with norketotifen for the underlying dermal condition. In another aspect, the patient has been previously treated with norketotifen for the underlying dermal condition but has stopped treatment with norketotifen prior to developing the dermal hypercytokinemia.

In an aspect, the norketotifen is administered topically or dermally as a rescue medication for immediate relief of the symptom of dermal hypercytokinemia.

In an aspect, the method further comprises diagnosing the dermal hypercytokinemia by determining from a lesional tissue skin biopsy from the patient a 2-fold or higher increase of one or more of TNFα, IL-4, IL-,5, IL-13, IFNG, INFγ, TGFβ-1 concentrations compared to normal tissue concentrations.

In a further aspect, the underlying dermal condition is a dermal viral infection, a dermal bacterial infection, a dermal fungal infection, atopic dermatitis, psoriasis, chronic urticaria, or a combination thereof.

Norketotifen is a potent, non-steroidal anti-inflammatory agent. In addition to its anti-inflammatory activity, norketotifen expresses anti-microbial effects against several types of bacteria, fungi and mold that may have infected the skin of patients suffering from dermal diseases.

Norketotifen is also a highly effective inhibitor of the release of cytokines and chemokines, such as those shown in Example 2. Advantageously, norketotifen is a small molecule and administration of norketotifen can provide for oral or topical administration and at very significant cost savings when compared with monoclonal antibodies. In addition, norketotifen is not a steroid and is therefore free from the adverse steroidal effects.

The term “human TNFα” (abbreviated herein as TNF) refers to a human cytokine that exists as a 17 kD secreted form and a 26 kD membrane associated form, the biologically active form of which is composed of a trimer of noncovalently bound 17 kD molecules. The structure of TNFα is described in the art (Tumor Necrosis Factor; Wikipedia). The term human TNFα is intended to include recombinant human TNFα, which can be prepared by standard recombinant expression methods or purchased commercially. Canine TNFα is closely related to human TNFα.

An advantage of using norketotifen for the treatment of dermal hypercytokinemia in a patient, e.g., a human or canine patient, suffering from a dermal microbial infection is that the risk for adverse corticosteroidal effects will be decreased or even avoided. Unlike corticosteroids which typically must be dosed at the lowest dose possible, norketotifen has surprisingly low toxicity and may be used in high oral doses and high topical concentrations (Example 4). Importantly, oral administration of norketotifen has been found to result in surprisingly high dermal concentrations of the drug. Example 3.

Thus, the use of norketotifen avoids numerous adverse effects of corticosteroids such as immunosuppression, adrenal gland atrophy; cataracts; facial hair growth; glaucoma; growth retardation in children; headache; high blood pressure; increased blood glucose and loss of diabetes control; loss of potassium; menstrual irregularity; muscle weakness; obesity; osteoporosis; puffiness of the face (moon face); slow wound healing; sodium and fluid retention causing edema and weight gain; thinning and easy bruising of the skin; ulcers in the stomach and duodenum; and other adverse steroidal effects.

The methods described herein relate to the prevention and treatment of dermal hypercytokinemia in a human and canine patients suffering from dermal viral, dermal bacterial and/or dermal fungal infections by orally administering, intravenously administering, subcutaneously administering, intramuscularly injecting, topically administering or dermally administering to the patient a therapeutically effective amount or concentration of norketotifen as a free base, an isomer, an isomeric mixture, a prodrug or a pharmaceutically acceptable salt thereof. In an aspect, the compound is racemic norketotifen.

Of particular note, after oral administration, norketotifen is rapidly absorbed and is surprisingly distributed to the skin, where the concentration of norketotifen can reach concentrations that are several times higher than the plasma concentration. Importantly, it was also found that the dermal residence time for norketotifen was significantly longer in the skin than in plasma as shown in Example 3.

When compared with the plasma drug concentrations, the skin concentration of norketotifen under laboratory conditions was up to 16 times higher than the plasma concentration. Also, the duration of the high concentration of norketotifen in the skin (dermal half-life) was up to 168 hours (one week) while plasma half-life of NK was 10 to 12 hours). Patients who suffer from dermal disorders such as atopic dermatitis, psoriasis or chronic urticaria will express multiple cytokines in their skin and may suffer from dermal hypercytokinemia. Leading experts have called an auto-immune dermal skin disease a product of a cytokine storm and the same investigators pointed out that inhibition of a single cytokine—even if it is TNFα—may not evoke significant activity to inhibit hypercytokinemia. However, norketotifen is the most potent mast cell inhibitor ever described and is simultaneously inhibiting the release of multiple cytokines is shown in Example 2. The unfortunate term “mast cell inhibitor” refers not only to mast cells, but to inhibition of the release of messenger proteins (such as cytokines and chemokines) from a variety of granulocytes.

As used herein, the term norketotifen refers to norketotifen free base, an isomer, an isomeric mixture, a prodrug, or a pharmaceutically acceptable salt thereof. RS-norketotifen refers to racemic norketotifen. In an aspect, the term norketotifen stands for RS-norketotifen hydrogen fumarate. Norketotifen is an achiral molecule, but has two atropisomers, S-norketotifen and R-norketotifen, as has previously been described by Aberg et al. in U.S. Pat. Nos. 7,226,934 and 7,557,128.

Isomeric norketotifen can be used, but the current inventors have found that isomeric norketotifen is rapidly racemized after administration to humans or to dogs. Example 3.

As explained in U.S. Pat. Nos. 7,226,934 and 7,557,128, norketotifen had significant sedative effects when studied in an art-accepted mouse model of sedation, and further, the sedative effects were attributed to the R-isomer. It was thus proposed that only the S-isomer could be administered without sedative side effects. It has later been found by the current inventors that orally administered RS-norketotifen is free from sedative side effects in dogs (U.S. Pat. No. 8,557,846) and in humans (U.S. Pat. Nos. 9,138,431 and 9,345,697). Therefore, unlike for ketotifen, no dose-limiting sedative adverse effects exist for norketotifen, even after high oral doses of norketotifen to humans or dogs.

Norketotifen can be made using methods known in the art, as described in U.S. Pat. No. 3,682,930, the disclosure of which is hereby incorporated by reference for its teaching of the synthesis of norketotifen.

A prodrug of norketotifen is defined as a compound that after administration to a mammal can be converted to norketotifen. Prodrugs of norketotifen include N-substituted hydroxyalkyl or carboxyalkyloxyalkyl analogs thereof. Examples of such molecules are described in U.S. Pat. No. 6,207,683. Thus, prodrugs of norketotifen include molecules of the formula

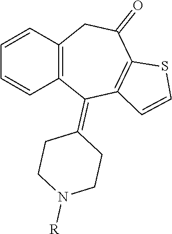

wherein R is for example hydroxy-C₂-C₆ alkyl or carboxy-C₁-C₆alkoxy-C₁-C₆alkyl. Additional prodrugs include substituents at the 8-position, the 10-position and/or in the 12 to 17 positions and/or inclusion of substituents on various positions on the piperidine ring.

As used herein, the terms “pharmaceutically acceptable salts” or “a pharmaceutically acceptable salt thereof” refer to norketotifen salts, which have been prepared from pharmaceutically acceptable non-toxic acids. Exemplary pharmaceutically acceptable acid as for the compound of the present invention include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride salt and the hydrogen fumarate salt as well as the free base of norketotifen are particularly preferred.

Embodiments disclosed herein provide for the oral and parenteral administration of norketotifen—such as dermal, transdermal, rectal and vaginal administration of norketotifen or its pharmaceutically acceptable acid addition salts thereof.

In an aspect, the norketotifen is administered orally in the form of a tablet, a capsule, or a syrup. In another aspect, the method further comprises co-administering none or more therapeutically active agent, selected from a group consisting of monoclonal antibody, a corticosteroid, a Jason kinas inhibitor, or combinations thereof.

The dosage of NK to human and canine patients will follow the recommendations by the local regulatory authorities.

Currently the recommendations by the inventors are that the oral dosage of norketotifen to a human patients suffering from dermal diseases shall start with a daily oral loading dose of from about 4 mg to about 30 mg of racemic norketotifen for 3 to 14 consecutive days, followed by a daily maintenance dose of NK, of 0.5 mg to 20 mg once or more times daily. The maintenance dosing will continue for as long as the human patient is suffering from the underlying disease. Canine patients may need oral loading doses of 4 mg/kg to 20 mg/kg of NK for 4 to 14 days, followed by a daily maintenance dose of 0.5 mg/kg to 10 mg/kg for as long time as the canine patient carries the underlying disease. In an aspect, the norketotifen is administered as a maintenance dose one to six times weekly.

Pharmaceutical compositions for oral administration of solid dosage forms of norketotifen include capsules, tablets, and liquid dosage forms, such as for example a syrup. In solid dosage forms, the therapeutically active compounds may be mixed with one or more pharmaceutically acceptable excipients or carriers (such as for example fumarate, sodium citrate, dicalcium phosphate), fillers (such as for example starch, lactose, sucrose, glucose, mannitol, silicic acid), binders (such as for example alginates, carboxymethylcellulose, gelatin, polyvinylpyrrolidone, sucrose, acacia), humectants (such as for example glycerol), solution retarding agents (such as for example paraffin), disintegrating agents (such as for example agar-agar, calcium carbonate, starch, alginic acid, silicates, sodium carbonate), absorption accelerators (such as for example quaternary ammonium compounds), wetting agents (such as for example cetyl alcohol, glycerol monostearate), absorbents (such as for example kaolin, bentonite clay), lubricating agents (such as for example talc, calcium stearate, magnesium stearate, polyethylene glycols, sodium lauryl sulfate), and/or other excipients, such as for example buffering agents. Solid forms of capsules, granules, pills, and tablets can have coatings and/or shells (such as for example enteric coatings) known in the art. The compositions may also be designed to release the active ingredient(s) in a certain part of the gastrointestinal tract or in a controlled release, slow-release or in a delayed-release manner. The active compound(s) can also be microencapsulated with one or more of the above-mentioned excipients or other suitable excipients.

Liquid dosage forms for oral administration of norketotifen include pharmaceutically acceptable emulsions, solutions, suspensions, syrups and elixirs. The liquid dosage form may also contain commonly known diluents (such as for example water, other solvents, solubilizing agents), emulsifiers, such as for example ethanol, isopropyl alcohol, ethyl carbonate, ethyl acetate, benzyl alcohol, benzyl benzoate, propylene glycol, butylene glycol, dimethyl formamide, oils, oleic acid, glycerol, polyethylene glycols, sorbitan, fatty esters, and mixtures thereof.

The actual dosage levels of active ingredients in the pharmaceutical compositions disclosed herein may be varied so as to obtain the desired therapeutic effect. Thus, the amount of drug used varies and will depend on factors such as the administration form, the severity of the disease, the frequency of dosing, and other circumstances (such as general health, body weight, age, etc.) known to the patient, the caregiver of the patient and/or the caring physician.

In an aspect, the norketotifen can be orally or topically/dermally administered. Transdermal administration is also possible since norketotifen has been found to rapidly be absorbed into the skin and across the skin.

The therapeutically effective oral doses of norketotifen useful for treating human patients with dermal conditions will be determined by the caring physician and are generally 1 mg to 50 mg (calculated as norketotifen free base), dosed orally as the free base or as a salt, such as for example the hydrochloride or mesylate salts or the hydrogen fumarate salt, once, twice or more times daily. In one embodiment, the treatment is once daily dosing. The therapeutically effective dose may be administered less than once daily, such as for example one to six times weekly, wherein administration is over at least a one-week period, as determined by the patient, the caretaker of the patient and/or the caring physician. In an aspect, the therapeutically effective amount of norketotifen, an isomer, an isomeric mixture, a prodrug or a pharmaceutically acceptable salt thereof for relief of dermal hypercytokinemia is administered topically or dermally at a concentration of about 0.1 percent to about 10 percent of norketotifen, calculated as norketotifen free base and administered one or more times daily. High doses of NK may be needed when NK is used as non-steroidal rescue medication for patients suffering from severe hypercytokinemia.

In a dosing regimen, the method includes orally administering, intravenously administering, subcutaneously administering, intramuscularly injecting, topically administering or dermally administering to the patient a therapeutically effective amount of a daily loading dose of the norketotifen, wherein the daily loading dose to the patient is administered from 3 consecutive days to 14 consecutive days or until a norketotifen-induced reduction in the symptom of dermal hypercytokinemia is observed, followed by orally administering, intravenously administering, subcutaneously administering, rectally administering, intramuscularly injecting, topically administering or dermally administering to the patient a therapeutically effective amount of a daily maintenance dose of the norketotifen, wherein the daily loading dose and the daily maintenance dose are about 1 mg to 30 mg per day, and wherein the loading dose is at least two times greater than the maintenance dose.

When the patient is a canine patient, the loading dose is 4 mg/kg to 20 mg/kg for 4 to 14 days, and the maintenance dose is 0.5 mg/kg to 10 mg/kg once or more times daily.

In an aspect, the norketotifen is administered as a maintenance dose one to six times weekly.

In addition to the use of norketotifen as single-drug medication in human and canine patients, embodiments disclosed herein also provide methods for co-administration of norketotifen with a corticosteroid. Thus, as an example, norketotifen can be administered orally and the co-administered drug may also be administered orally or topically/dermally. Furthermore, norketotifen can be administered topically/dermally in combination with oral documentation to inhibit the release of cytokines from granulocytes or other cells or carriers. Thus, as an example, norketotifen may be administered orally once daily, while norketotifen in addition to the oral administration may be administered topically on the skin once or multiple times daily.

Exemplary underlying dermal conditions include a dermal viral infection, a dermal bacterial infection, a dermal fungal infection, atopic dermatitis, psoriasis or chronic urticaria.

### Psoriasis (PSO)

There are several forms of human psoriasis. Common forms of psoriasis are plaque psoriasis (the most common form of psoriasis, which causes dry, raised, red skin patches often called lesions), arthritic psoriasis (causes one or more painful joints in addition to dermal psoriatic symptoms), guttate psoriasis (most often affecting young adults and children; caused by initial bacterial infection), inverse psoriasis (mainly affecting skin folds; caused by initial fungus infection) and pustular psoriasis (rare form of psoriasis; causes clearly defined pus-filled lesions on palms of hands or the soles of feet). All forms of psoriasis are included herein under the term psoriasis.

About 8 million Americans have psoriasis and worldwide 125 million people suffer from psoriasis. Psoriasis is not a cosmetic problem. Nearly 60 percent of people with psoriasis reported their disease to be a large problem in everyday life.

Psoriasis is not generally thought of as a lethal disease, although a study from the UK concluded that the life expectancy for people with severe psoriasis is shorter than the lives of people without psoriasis. NK can be used as nonsteroidal rescue medication by patients suffering from severe psoriasis-related hypercytokinemia.

The following cytokines have been reported to be over-expressed in lesional skin of psoriasis patients: TNFα, IL-1, IL-2, IL-6, IL-8, IL-10, IL-12, IL-13, IL-17, IL-18, IL-22, IL-23, IL-36, IL-37, INFα, MCP-1 and MIP-1a. To date, most of these cytokines (Example 4) have been tested herein and the release of all tested cytokines were inhibited by norketotifen with statistical significance.

NK is particularly well suited for the treatment of psoriasis-related cytokine storms, which are caused by numerous cytokines and are called psoriasis-related hypercytokinemia. NK is an anti-inflammatory drug (Example 1) that potently inhibits the release of pro-inflammatory cytokines from activated mast cells, activated eosinophils, activated leucocytes (Example 4) and other cells, such as keratinocytes that constitute 90 percent of all epidermal skin cells. NK can also be administered to patients at risk for development of psoriasis-related hypercytokinemia, such as patients expressing one or more early symptoms of psoriasis-related hypercytokinemia. Local lesions and local pruritus are symptoms of psoriasis and intensification of psoriatic lesions and/or intense pruritis are symptom of psoriasis-related hypercytokinemia.

Numerous animal models of psoriasis exist. The Testerman Imiquimod model (Example 3) is considered to be the most predictive and most reliable model because imiquimod is causing the release of numerous cytokines that are identical to those released in psoriasis patients. The following cytokines are known to be involved in psoriasis, and all of them were significantly inhibited by NK:TNFα, IL-lb, IL-2, IL-4, IL-6, IL-10, IL-13, INFα, MCP-1, MIP-1α.

### Atopic Dermatitis (AD)

Through its inhibitory effects on the release of pro-inflammatory cytokines, norketotifen will reduce the symptoms of dermal hypercytokinemia after topical/dermal applications of gels, creams or ointments containing norketotifen in concentrations ranging from about 0.5 to about 10 percent of norketotifen, calculated as free base, but also used as a salt. Those skilled in the art of topical drug application may realize that NK can also be delivered as a cataplasm (poultice) dermal delivery system that will offer rapid transdermal delivery of norketotifen in high (up to 10 percent if solubilizers such polyethylene glycol are used. Due to its significant accumulation of NK in the skin and its slow elimination from the skin (Example 5), norketotifen is likely to express dermal antimicrobial activity after systemic/oral administrations of high oral doses such as 10 to 20 milligrams, one or multiple times daily to patients suffering from dermal infections. Doses in the higher range may be needed as nonsteroidal rescue medication by patients suffering from severe AD-related hypercytokinemia. Norketotifen is a potent and dose-dependent inhibitor of the release of pro-inflammatory cytokines that are involved in AD-related dermal hypercytokinemia (Example 2). Norketotifen is of these reasons well suited as medication for the treatment of patients suffering from AD-related dermal hypercytokinemia or for patients expressing early indication for risk for development of AD-related hypercytokinemia. Early indications for the development of AD-related hypercytokinemia include the appearance of multiple new lesions and/or the patient complaining of unusually severe pruritus. The following cytokines are known to be involved in AD-related hypercytokinemia: IL-4, IL-6, IL-8, IL-13 and TNFα. All of them were tested by the current inventors and the release of all of them from granulocytes were inhibited by NK (Example 2).

### Chronic Idiopathic Urticaria/Chronic Pruritus

Nomenclature: Urticaria is a disease, characterized by transient pruritic wheals. Pruritus is not a disease, but a symptom or a sensation. The classical definition of pruritus is “an irritating sensation that makes you want to scratch.” Urticaria is often diagnosed in dogs where it is often referred to as “hives” or “rashes.”

Urticaria has numerous etiologies. Various receptors are involved such as TRPA1, TRPV1, PAR-2, gastrin-releasing peptides, Mas-related G-proteins, histamine receptors, nerve growth factor and Substance P. It is also known that itch is caused by a complex interface between skin, keratinocytes, cutaneous nerve fibers, pruritogenic molecules and the peripheral and central nervous systems. In recent years, pruritus-research has centered on specific cytokines (mainly IL-31) that has been shown to be involved in pruritus and a monoclonal antibody for dogs is targeting IL-31 (Cytopoint®, Zoetis) and has therapeutic effects in many pruritic canine patients.

Cytokines involved in chronic urticaria are TNFα, IL-1, IL4, IL-5, IL-6, MCP-1, MIP-1a, IL-31, IL-13 and IFNγ. The current inventors have tested these cytokines (Example 2) and the release of all the tested cytokines from human leucocytes were inhibited by norketotifen with statistical significance (p<0.05).

Norketotifen has also been found to inhibit the pruritic effect of IL-31, thereby demonstrating inhibition of IL-31-induced pruritus by norketotifen (Example 7).

In specific aspects, the norketotifen inhibits the release from granulocytes of IL-4, IL-6, IL-10, TNF-α, INF-γ, IL-13, IL-1β, IL-2, MCP-1, M1Pa, or combinations thereof.

In a specific aspect, the patient is a human patient suffering from virus-initiated hypercytokinemia, atopic dermatitis-related hypercytokinemia, psoriasis-related hypercytokinemia, urticaria -related hypercytokinemia. In another aspect, the patient is a human patient suffering from bacterial-initiated hypercytokinemia, fungus-initiated hypercytokinemia, or fungus and bacteria-related hypercytokinemia. In another aspect, the patient is a human patient suffering from plaque psoriasis, arthritic psoriasis, guttate psoriasis, inverse psoriasis, or pustular psoriasis.

In a further aspect, the patient is a canine patient suffering from virus-initiated hypercytokinemia, bacterial-initiated hypercytokinemia, fungus-initiated hypercytokinemia, or combined fungus and bacteria-related hypercytokinemia. In a yet further aspect, the patient is a canine patient suffering from atopic dermatitis-related hypercytokinemia, or urticaria-related hypercytokinemia.

### EXAMPLES

The current invention is further illustrated by the following non-limiting examples.

### Example 1

### Imiquimod (IMQ) Induced Psoriasis in Mice

### Objective

The objective of this study was to assess the ability of NK to inhibit the IMQ-induced psoriasiform hypercytokinemia in mice. Topically applied IMQ is known to induce a psoriasis-like Cytokine Storm in mice by releasing a large number of cytokines, thereby causing a disease (hypercytokinemia) that is closely similar to psoriasis in human patients.

### Methodology

Female BALB/c mice were used (8-11 weeks old; 4 animals/group). A psoriasiform skin condition was induced by repeated applications of an IMO-cream on the pre-shaved skin of the back of mice for 6 days, using Aldana 5% cream (IMQ-cream), at a dose of 62.5 mg per mouse, which is equal to 3.125 mg active per mouse. Starting from day 3 of dosing the skin was scored for Redness, Thickness and Scaling using a scoring system on the scale of 0 to 4, where 0=none; 1=slight; 2=moderate; 3=marked; and 4=very marked. The cumulative scores (erythema plus scaling plus thickness) served as a measure of the severity of dermal, psoriasis-like inflammation. On Day 6, about 22 hours after previous day IMQ-cream application, mice from each group were observed for 30 minutes for scratch bouts (itching). A scratch bout was defined as one or more rapid back-and-forth hind paw motions directed toward and contacting the IMQ-treated area.
Group 1: (IMQ Positive Control Group): 4 mice were treated as described above.
Group 2: 4 mice were given NK 10 mg/kg/day, PO, starting 3 days before the first application of IMQ.
Group 3: same treatment as Group 2, but the dose of NK was 20 mg/kg/day.
Group 4: same treatment as Group 3, but the dose of NK was 30 mg/kg/day.

TABLE 1
EFFECTS OF NK AGAINST INTENSE PRURITUS

| Group Treatment | Scratching bouts | SEM |
|---|---|---|
| IMQ 5%-topically applied (control) | 40.23 | 31.62 |
| IMQ + NK 10 mg/kg-oral dose | 7.0 | 1.58 |
| IMQ + NK 20 mg/kg-oral dose | 4.25 | 1.03 |

### Conclusions

There was a dose-dependent effect of NK on scratching in mice, although the effect was not statistically significant (p>0.05), because one positive control mouse was an obvious outlier. The outlier was not removed since there may be a biological variation of IMQ-induced pruritus. This result is significant because pruritus related to hypercytokinemia is indeed extremely intense and it was previously assumed that rescue medication with high-dose corticosteroids was the only effective medication for patients who were in need of rescue medication.

TABLE 2
EFFECTS OF NK AGAINST IMIQUIMOD-INDUCED
INTENSE PSORIASIS IN MICE. SUMMARY OF SCORES

| | Mean Scores | | | | Statistical |
|---|---|---|---|---|---|
| | of Skin Lesions | | | Total | signif- |
| | | Thick- | | Scores ± | icance |
| Group | Redness | ness | Scaling | SEM | (p)) |
| IMQ 5% | 2.00 | 1.38 | 1.25 | 4.63 ± 0.56 | — |
| IMQ + NK | 0.75 | 0.5 | 0.25 | 1.5 ± 0.65 | <0.01 |
| 10 mg/kg- | | | | | |
| oral dose | | | | | |
| IMQ + NK | 0.5 | 0 | 0 | 0.5 ± 0.29 | <0.001 |
| 20 mg/kg- | | | | | |
| oral dose | | | | | |
| IMQ + NK | 0.33 | 0 | 0 | 0.33 ± 0.33 | <0.001 |
| 30 mg/kg- | | | | | |
| oral dose | | | | | |

IMQ—imiquimod 5% cream, applied daily and topically.
NK = Norketotifen dosed orally at 10, 20 or 30 mg/kg/day

### Calculations

ANOVA (Dunnett's Method)—positive control (IMQ) vs test groups.

### Comments

The methodology used here was the van der Fits (2009) method with minor modifications.

### Conclusions

In this study, norketotifen almost completely inhibited all psoriasis-related cytokine storm as evidenced by the antipruritic activity of NK in Table 1 and the NK-reduced redness, thickness and scaling scores in Table 2.

### Example 2

### Cytokine Inhibition by Norketotifen (NK)

Objective:

The objective of this study was to assess the ability of norketotifen to inhibit cytokine release from stimulated human leucocytes.

### Methodology:

The study was conducted in two phases. The first phase included optimization of the test methodology and including dose-range finding studies, using buffy coats from 3 human donors, stimulated with phytohemagglutinin (PHA). Following optimization of the test conditions, a final protocol was issued. In the ensuing Main Study, white blood cells (buffy coats) from 5 healthy human donors were stimulated with PHA, 5 μg/ml. The concentrations of the test article and the duration of exposure were optimized to obtain acceptable dose-response ratios. Thus, the inhibitory effects of three concentrations of NK and one concentration of ketotifen were evaluated in the Main Study. The buffy coats containing white blood cells from 5 male human donors were resuspended in assay media and were pre-incubated for 30 min with either vehicle/saline, ketotifen 4.3 μg/ml (10 μM) and norketotifen 0.4 μg/ml, 4.1 μg/ml and 41 μg/ml (1.0; 10 and 100 μM). Following these preincubations, the white blood cells were stimulated for 16 hours with PHA, 5 μg/ml. The cell system supernatants were then harvested and stored frozen pending analyses.

### Results

Table 3 demonstrates potent and dose-depending inhibitory effects of norketotifen on the release of pro-inflammatory cytokines and chemokines.

TABLE 3
INHIBITION BY NORKETOTIFEN AND KETOTIFEN OF PHA-
INDUCED RELEASE OF SELECTED CYTOKINES AND
CHEMOKINES FROM HUMAN LEUCOCYTES (BUFFY COATS)

| Cytokine (C)) | Ketotifen (%) | Norketotifen (%) | | |
|---|---|---|---|---|
| Chemokines (Ch) | 10 uM | 1 uM | 10 uM | 100 uM |
| IL-1β (C) | 0.00 | 0.00 | 16.20² | 57.00¹ |
| IL-6 (C) | 0.00 | 0.00 | 33.13¹ | 88.16¹ |
| TNFα (C) | 0.00 | 4.54 | 42.19² | 89.13¹ |
| IL-4 (C) | 0.00 | 0.00 | 14.32¹ | 98.27¹ |
| IL-2 (C) | 0.00 | 0.00 | 46.63¹ | 93.47¹ |
| IL-8 (Ch) | 19.99 | 10.29² | 34.36¹ | 78.29¹ |
| IL-10 (C) | 0.00 | 12.71 | 24.80² | 90.40¹ |
| IL-13 (C) | 0.00 | 0.00 | 34.49¹ | 74.25¹ |
| INF-γ (C) | 0.00 | 0.00 | 16.54² | 71.56¹ |
| MCP-1(Ch) | N/A | 2.04 | 14.35² | 84.52¹ |
| MIP-1a(Ch) | N/A | 9.53² | 25.94² | 90.99¹ |

Values marked ¹denote statistically significant difference (p < 0.05) when compared with vehicle.
Values marked ²denote decrease in concentration that was not statistically significant; (p > 0.05).
The vehicle/saline had no inhibitory effects on PHA-induced cytokine or chemokine release.
(C) = Cytokine (Ch) = Chemokine

### Conclusions

Norketotifen dose-dependently inhibited the release of cytokines and chemokines from human white blood cells. No statistically significant inhibition of cytokine release was expressed by ketotifen.

### Footnote

The concentration of the test articles had to be high in this ex vivo study since the test kit used a high concentration of the agonist PHA (5 μg/ml) and a long exposure time (16 hrs).

In particular, cytokines IL6, TNFα, IL-2 and IL1β may act as triggers of cytokine storms.

### Example 3

### Dermal and Plasma Concentrations of NK After Oral Dosing to Dogs

### Purpose

The purpose was to determine drug concentrations and calculate pharmacokinetics in the skin and plasma of dogs during and following daily oral administration of S-NK-HF (the S-isomer of norketotifen hydrogen fumarate) for four weeks.

### Methods

Five male beagle dogs, weighing 11.2-13.9 kg (2-4 years old) were included in the study. All animals were administered p.o. doses of the test article 8.0 mg/kg/day as a hydrogen fumarate salt, equal to 5.6 mg/kg/day of the free base. A single isomer of NK was used, since an objective of this study was also to assess the racemization of the NK isomers. The animals were dosed once daily for four weeks followed by daily observations and tissue sampling for an additional two-week washout period.

Multiple plasma samples and skin biopsies were taken from each dog on Day 1 and Day 28 of drug administration. The plasma and skin samplings were performed at pre-dose, and at 2, 6, 12, and 24 hours post-dose. Plasma and skin samples were also taken intermittently at predetermined intervals during the 28-days dosing period and up to the last day of the study, which was Day 42. Blood samples were taken from v. cephalica antebrachii. Skin biopsies were taken from the area between the mid ventral to lateral abdominal areas, using a 6 mm skin biopsy punch. Subcutaneous fat deposits were carefully trimmed from the skin samples and the skin samples were then weighed. All plasma and skin samples were analyzed using LC/MS/MS methodology.

### Results

As shown in the table below, AUC, T1/2 and MRT of both S- and R-NK are significantly increased in the skin as compared with the same parameters in plasma.

TABLE 4
PK PARAMETERS AFTER ORAL DOSING OF S-NK,
8.0 MG/KG/DAY FOR 28 DAYS TO DOGS (N = 5)

| | S-NK | S-NK | R-NK | R-NK | RS-NK | RS-NK |
|---|---|---|---|---|---|---|
| PK Parameter | Plasma | Skin | Plasma | Skin | Plasma | Skin |
| AUC_{∞} | 1627 | 25710 | 1658 | 20376 | 3286 | 54187 |
| ng*hr/mL or g | | | | | | |
| T_{1/2} (hrs) | 10.9 | 162.7 | 7.7 | 157.0 | 10.5 | 167.6 |
| MRT (hrs) | 11.3 | 159.0 | 14.1 | 159.1 | 12.8 | 169.3 |

AUC_{∞} = Area under the plasma concentration (or skin concentration) curves vs. time T_{1/2} = Plasma or skin half-life; MRT = Mean residence time; NK = norketotifen.

### Conclusions

RS-norketotifen was rapidly formed after oral administration of 5-norketotifen. Both S- and R-isomers were completely and very rapidly racemized in dogs. The plasma half-life of RS-NK was 10-11 hours which confirms previous plasma half-lives in dog studies. The long dermal half-life of norketotifen in skin of dogs (one week) may allow for less than once daily oral administration of norketotifen.

### Example 4

### Skin Irritation Study in Göttingen Mini Pigs

### Objective

To assess possible skin irritation following repeated dermal application of racemic norketotifen as a hydrogen fumarate salt (NK-HF) to Gottingen Minipigs.

### Method

Standard creamy polyethylene-glycol (PEG400/PEG4000; herein called PEG) formulations containing 0% (vehicle control), 5% or 10% of RS-norketotifen hydrogen fumarate (NK-HF) were prepared and applied once daily for four consecutive days to three marked areas (3 inches×3 inches) of the shaved skin of Gottingen minipigs pigs (2M+2F, 7-8 months; 9.7-11.6 kg). The volumes used for each application were 3.5 mL). Four animals were used in this study. The number of animals was intended to satisfy regulatory guidelines, to meet general scientific principles and to meet Bridge Pharma's animal-sparing commitments. The vehicle and the test articles were applied once daily for four consecutive days, and erythema (redness), edema (swelling) and eschar (scab) were assessed by the Study Director, who personally assessed and scored the appearance of the skin before the daily cream applications and four hours thereafter on Days 1 through 4. Additional inspections and scorings were performed on Day 5, twenty-four hours after the last (Day 4) applications of the vehicle and test articles.

### Results

Dermal applications, once daily for four consecutive days of creamy PEG formulations of NK-HF 5% and NK-HF 10% did not induce any irritation in any of the Göttingen minipigs during or after the dermal applications.

### Conclusions

When used topically in the clinic, NK-HF is currently assumed to be used at concentrations of about one or two percent. In the current study NK-HF was applied topically to the shaved skin of minipigs at concentrations of 5 percent and 10 percent in order to establish a wide margin of safety.

### Example 5

### Effects of NK on IL-31-Induced Pruritus In Mice

### Objective

To determine if NK inhibits IL31-induced pruritus in mice.

### Background and Methodology

There is interest in IL31 as a cytokine involved in pruritus. The scientific introduction of the canine AD-drug oclacitinib was focused on the effects on IL31-induced pruritus.

Studies have now been performed, using murine IL31 as a pruritogen. Mice were dosed orally with a test article exactly 60 minutes before murine IL31 at a dose of 1.0 μg/30 gram body weight was injected subcutaneously into a previously depilated area on the rostral part of the back of mice. IL31 was dissolved in phosphate buffered saline (PBS) with 0.1% bovine serum albumin (BSA). The injected volume was 0.1 ml/mouse. The bouts of scratching were counted for 120 min, starting 30 min after the injection of IL31. The bouts of scratching were counted by trained laboratory personnel who were unaware of the pretreatment of the animals. Oclacitinib (Apoquel®) is a Janus kinase inhibitor that is the worldwide market leader for oral treatment of atopic dermatitis in dogs.

### Results

TABLE 5
ANTIPRURITIC ACTIVITY IN MICE;
IL31-INDUCED (SC) PRURITUS

| Test | | Pruritic Bouts | Protection |
|---|---|---|---|
| Article | N | Mean ± SEM | (%) |
| Vehicle | 8 | 203 ± 45 | — |
| Norketotifen | 8 | 93 ± 10 * | 54 |
| HF 10 mg/kg | | | |
| Oclacitinib | 8 | 116 ± 29 | 43 |
| maleate | | | |
| 10 mg/kg | | | |

* means P ≤ 0.05 when compared with Vehicle.
HF = hydrogen fumarate (salt)
10.0 mg/kg of norketotifen HF salt = 7.18 mg/kg of norketotifen free base
10.0 mg/kg of oclacitinib maleate salt = 7.44 mg/kg of oclacitinib free base

### Conclusion

NK decreased IL-31-induced pruritus at the dose tested.

### Justification of the Model

Activated dermal IL-31 receptors (IL-31R) on dorsal root ganglia (DRG) are important links between the pruritic cytokine IL-31 and the generation of pruritic action-potentials on DRGs in patients suffering from often unbearable pruritus as part of psoriasis-related, AD-related or urticaria-related cytokine storm syndromes. NK-induced inhibition of IL-31R may inhibit IL-31-induced pruritus.

### Example 6

### Exemplary Oral Dosage Formulation

Norketotifen may be administered orally as a tablet, a capsule, or as a syrup. To make tablets, norketotifen as free base, or as a salt, an isomer or a prodrug thereof is blended with lactose and cellulose until a uniform blend is formed. The blue lake is added and further blended. Finally, the calcium stearate is blended in, and the resulting mixture is compressed into tablets using for example a 9/32-inch (7 mm) shallow concave punch. Tablets of other strengths may be prepared by altering the ratio of active ingredient to the excipients or to the final weight of the tablet. Those skilled in the art realize that formulations can also be administered to the patient in the form of for example a capsule, a cream, an ointment or a liquid formulation. Both norketotifen salts and norketotifen free base can be formulated as tablets.

TABLE 8
TABLET FORMULATIONS

| Ingredient | Amount per tablet | | Amount per batch |
|---|---|---|---|
| Norketotifen (NK) | 8 | mg | 800 g |
| Microcrystalline cellulose | 24 | mg | 2400 g |
| Lactose | 56 | mg | 5600 g |
| Calcium stearate | 1.4 | mg | 140 g |
| FD&C Blue #1 Lake | 0.03 | g | 3 g |

As used herein, the terms “pharmaceutically acceptable salts” or “a pharmaceutically acceptable salt thereof” refer to norketotifen salts, which have been prepared from pharmaceutically acceptable non-toxic acids. Exemplary pharmaceutically acceptable acid as for the compound of the present invention include acetic, benzenesulfonic (besylate), benzoic, camphorsulfonic, citric, ethanesulfonic, fumaric, gluconic, glutamic, hydrobromic, hydrogen fumaric, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride salt and the hydrogen fumarate salt are particularly preferred.

The term “antimicrobial” as used herein refers to antibacterial, antifungal and anti-mold activities or effects.

The term microbe, microbial and similar refer to bacterial, viral, fungal, mold and/or other living forms.

The term “patient” as used herein refers to human patients and/or canine patients.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted.

As used herein, when referring to dosage amount, the term “about” includes amounts to ±10% of the recited value.

As used herein, the term “chronic administration” is defined as three or more consecutive days of administration. Acute administration of norketotifen refers to a single administration of the drug.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention shall not be limited to the particular embodiment disclosed as the best mode contemplated for carrying out this invention, but that the invention will include all embodiments falling within the scope of the appended claims.

1. A method of reducing the incidence and/or intensity of a symptom of dermal hypercytokinemia in a patient suffering from an underlying dermal condition who develops the dermal hypercytokinemia as a consequence of the underlying dermal condition, said method comprising orally administering, intravenously administering, subcutaneously administering, intramuscularly injecting, topically administering or dermally administering to the patient a therapeutically effective amount of norketotifen as a free base, an isomer, an isomeric mixture, a prodrug or a pharmaceutically acceptable salt thereof, wherein the symptom of dermal hypercytokinemia is an eruption of new dermal lesions accompanied by intense lesional and/or non-lesional pruritus.

2. The method of claim 1, wherein the patient has not been previously treated with norketotifen for the underlying dermal condition, or wherein the patient has been previously treated with norketotifen for the underlying dermal condition but has stopped treatment with norketotifen prior to developing the dermal hypercytokinemia.

3. The method of claim 1, comprising orally administering, intravenously administering, subcutaneously administering, intramuscularly injecting, topically administering or dermally administering to the patient a therapeutically effective amount of a daily loading dose of the norketotifen, wherein the daily loading dose to the patient is administered from 3 consecutive days to 14 consecutive days or until a norketotifen-induced reduction in the symptom of dermal hypercytokinemia is observed,
followed by orally administering, intravenously administering, subcutaneously administering, rectally administering, intramuscularly injecting, topically administering or dermally administering to the patient a therapeutically effective amount of a daily maintenance dose of the norketotifen,
wherein the daily loading dose is about 4 mg to 30 mg per day and the daily maintenance dose is about 0.5 mg to 20 mg per day, and wherein the loading dose is at least two times greater than the maintenance dose.

4. The method of claim 1, comprising orally administering, intravenously administering, subcutaneously administering, intramuscularly injecting, topically administering or dermally administering to a canine patient a therapeutically effective amount of a daily loading dose of the norketotifen, wherein the daily loading dose to the patient is administered from 4 consecutive days to 14 consecutive days or until a norketotifen-induced reduction in the symptom of dermal hypercytokinemia is observed,
followed by orally administering, intravenously administering, subcutaneously administering, rectally administering, intramuscularly injecting, topically administering or dermally administering to the canine patient a therapeutically effective amount of a daily maintenance dose of the norketotifen,
wherein the daily loading dose is 4 mg/kg to 20 mg/kg and the daily maintenance dose is 0.5 mg/kg to 10 mg/kg, administered one or more times daily, and wherein the loading dose is at least two times greater than the maintenance dose.

5. The method of claim 1, wherein the norketotifen is administered topically or dermally as a rescue medication for immediate relief of the symptom of dermal hypercytokinemia.

6. The method of claim 1, further comprising diagnosing the dermal hypercytokinemia by determining from a lesional tissue skin biopsy from the patient a 2-fold or higher increase of one or more of TNFα, IL-4, IL-,5, IL-13, IFNG, INFγ, TGFβ-1 concentrations compared to a normal tissue concentration.

7. The method of claim 1, wherein the underlying dermal condition is a dermal viral infection, a dermal bacterial infection, a dermal fungal infection, atopic dermatitis, psoriasis, chronic urticaria, or a combination thereof.

8. The method of claim 1, wherein the norketotifen is administered as a maintenance dose one to six times weekly.

9. The method of claim 1, wherein the norketotifen inhibits the release from granulocytes of IL-4, IL-6, IL-10, TNF-α, INF-γ, IL-13, IL-1β, IL-2, MCP-1, M1Pa, or a combination thereof.

10. The method of claim 1, wherein the norketotifen is orally administered in the form of a tablet, a capsule, or a syrup, or wherein the norketotifen is topically or dermally administered in the form of a gel, an ointment, a cream, or a cataplasm delivery system.

11. The method of claim 1, wherein a the therapeutically effective amount of norketotifen, an isomer, an isomeric mixture, a prodrug or a pharmaceutically acceptable salt thereof for relief of dermal hypercytokinemia is administered orally from about 1 mg to about 50 mg per dose of norketotifen, calculated as norketotifen free base and administered one or more times daily.

12. The method of claim 1, wherein a the therapeutically effective amount of norketotifen, an isomer, an isomeric mixture, a prodrug or a pharmaceutically acceptable salt thereof for relief of dermal hypercytokinemia is administered topically or dermally at a concentration of about 0.1 percent to about 10 percent of norketotifen, calculated as norketotifen free base and administered one or more times daily.

13. The method of claim 1, wherein the patient is a human patient suffering from virus-initiated hypercytokinemia, atopic dermatitis-related hypercytokinemia, psoriasis-related hypercytokinemia, or urticaria -related hypercytokinemia.

14. The method of claim 1, wherein the patient is a human patient suffering from bacterial-initiated hypercytokinemia, fungus-initiated hypercytokinemia, or fungus and bacteria-related hypercytokinemia.

15. The method of claim 1, wherein the patient is a canine patient suffering from virus-initiated hypercytokinemia, bacterial-initiated hypercytokinemia, fungus-initiated hypercytokinemia, or combined fungus and bacteria-related hypercytokinemia.

16. The method of claim 1, wherein the patient is a human patient suffering from plaque psoriasis, arthritic psoriasis, guttate psoriasis, inverse psoriasis, or pustular psoriasis.

17. The method of claim 1, wherein the patient is a canine patient suffering from atopic dermatitis-related hypercytokinemia, or urticaria-related hypercytokinemia.
